(19) United States
(12) Patent Application Publication
(10) Pub. No.: US 2014/0058267 A1
(43) Pub. Date: Feb. 27, 2014

(54) NON-INVASIVE CONTINUOUS DOPPLER MONITORING DEVICE FOR ARTERIAL BLOOD FLOW TO DISTAL BODY PARTS

(71) Applicants: Brad Eliot Kessler, San Diego, CA (US); Andrew Hammond, Portland, OR (US)

(72) Inventors: Brad Eliot Kessler, San Diego, CA (US); Andrew Hammond, Portland, OR (US)

(21) Appl. No.: 13/945,184

(22) Filed: Jul. 18, 2013

Related U.S. Application Data
(63) Continuation-in-part of application No. 12/852,797, filed on Aug. 9, 2010.
(60) Provisional application No. 61/232,433, filed on Aug. 9, 2009.

(51) Int. Cl. A61B 8/06

(52) U.S. Cl. CPC A61B 8/06
USPC 600/454

(57) ABSTRACT

A Doppler sensor for non-invasive continuous Doppler monitoring for arterial blood flow to a distal body part of a patient that employs a Doppler transducer holder for placement at the distal body part of the patient. The holder is adapted to allow the sensor to be inserted and removed, enabling a sterile sensor for each new use. The holder may be disposable after each use, may be provided with a cover for disposal of the cover after each use and re-use of the holder, and the sensor may be provided with a cover for sterile placement against the patient's body.

This application claims priority of U.S. provisional patent application 61/232,433, filed Aug. 9, 2009, entitled NON-INVASIVE CONTINUOUS DOPPLER MONITORING DEVICE FOR ARTERIAL BLOOD FLOW TO DISTAL BODY PARTS, and is a continuation-in-part of U.S. patent application 12852797 filed Aug. 9, 2010, entitlted NON-INVASIVE CONTINUOUS DOPPLER MONITORING DEVICE FOR ARTERIAL BLOOD FLOW TO DISTAL BODY PARTS.

### BACKGROUND OF THE INVENTION

This invention relates to monitoring of blood flow, and more specifically to a non-invasive blood flow monitoring device and method.

Many times during certain medical procedures, blood flow is restricted to purposely decrease blood flow and create a wanted blood clot, i.e. during femoral sheath removal post PTCA/Stent. With such blood flow restriction, it is important to ensure that distal body parts are receiving adequate blood flow. For example, in some procedures, an upper leg portion will be compressed or clamped to reduce arterial blood flow to the leg, to minimize bleeding during the procedures or post procedure. However, some blood flow must be maintained to ensure adequate supply to the extremities, to avoid tissue damage.

To ensure adequate blood flow is occurring while the compression/clamp is applied, current practice is to intermittently apply a Doppler monitoring device at a pulse point of the relevant distal body part, to determine if there is sufficient blood flow. Typically the blood flow is monitored in the posterior tibial artery, dorsalis pedis artery, or radial artery. The monitoring device includes a hand-held probe (transducer) and monitor which provides an audible representation of blood flow. The probe consists of, for example, an elongate probe body such as a pencil-like steel tube, or other probe body with contoured shape and materials for more comfortable hand-held use by medical personnel, with a Doppler chip placed flush with one end of the probe body, and sensor wires running from the Doppler chip, up the center and out the other side of the probe body. The probe wires are connected to a Doppler monitor device which transmits signals to the Doppler chip and reads sensed return signals sent back to the monitor from chip, converting the signals to, for example, an audio signal which the medical personnel can hear to determine that blood flow is sufficient.

Doppler pulses are sometimes difficult to find when using the traditional Doppler device, even though the pulse is known to be present. With intermittent monitoring, an MD/RN/tech has to relocate the pulse each time the probe is applied (which can be more complicated since blood flow has been reduced or might be insufficient to provide a pulse), and the intervals of monitoring must be sufficiently closely spaced to avoid long periods of insufficient blood flow. If a surgical procedure is underway, timing of the monitoring check points may not be practical relative to the timing needs of the surgical procedure.

Since the monitoring is only done periodically, the status of blood flow between monitoring points is unknown, and a positive flow status during the monitoring times may not accurately reflect the blood flow state between monitoring points.

To attempt to address some of the difficulties, intermittent monitoring, medical personnel time requirements and requiring relocating a pulse each time, some medical teams have attempted to tape the monitor probe to the patient at the location where the pulse is found. However, since the probe is designed to be hand-held and moved around to be positioned in a given location only very briefly, the probe configuration and shape, with the transducer at the tip end of a generally elongate probe body, makes it difficult to securely position and maintain the probe in the proper location and orientation such that it can properly detect the blood flow over extended periods.

Also, it is important that the efforts of positioning the sensor and monitoring not interfere with the medical procedure itself.

### SUMMARY OF THE INVENTION

In accordance with the disclosure a small, light, portable, easy to use/install device is provided so that a reduced number of personnel are required in medical procedures, with no or minimal interference with the medical procedure from the sensor configuration and attachment.

The subject matter of the present invention is particularly pointed out and distinctly claimed in the concluding portion of this specification. However, both the organization and method of operation, together with further advantages and objects thereof, may best be understood by reference to the following description taken in connection with accompanying drawings wherein like reference characters refer to like elements.

### BRIEF DESCRIPTION OF THE DRAWINGS

FIG. 1 is a top view schematically illustrating use of the system and method;

FIG. 2 is a perspective view of a particular embodiment of the sensor in accordance with the system and method;

FIG. 3 is an exploded perspective view of the sensor of FIG. 2;

FIGS. 4-9 are front angled, isometric, side, top, left side and bottom views of an improved grip Doppler transducer; and

FIGS. 10 and 11 are views of a removable sensor portion; and

FIG. 12 is a view of a holder with sterile removable cover.

### DETAILED DESCRIPTION

The system and method according to a preferred embodiment comprises a Doppler transducer with adhesive for attachment to a patient and connecting wires to connect to a Doppler monitor.

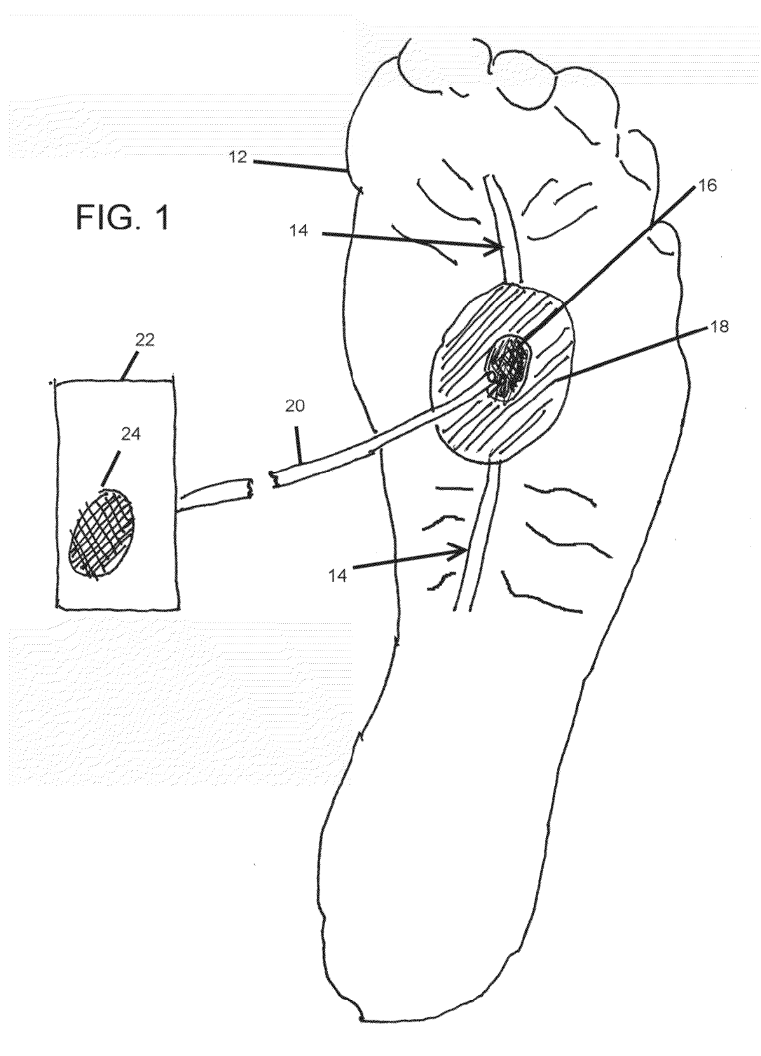

Referring to FIG. 1, a top view schematically illustrating use of the system and method, a patient's foot 12 is shown, with the location of the dorsalis pedis artery 14 visible. A Doppler transducer 16 is positioned on the outside of the foot, maintained in position until later removal by use of adhesive conductive gel 18. Cable 20 provides a connection between Doppler monitor 22 and the Doppler transducer 16, such that the monitor can interact with the transducer to produce audio, via speaker 24, representing sensed blood flow.

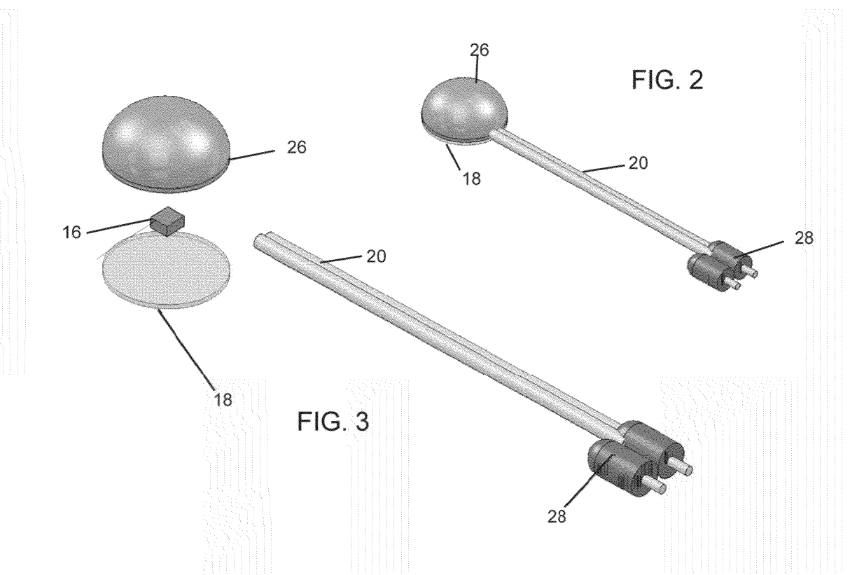

FIG. 2 is a perspective view of a particular embodiment of the sensor in accordance with the system and method, wherein a semispherical sensor overmold 26 is provided to contain the transducer therein and carry the adhesive for attachment to the patient. Adhesive conductive gel 18 is positioned on the bottom of the sensor overmold, and sensor cable 20 extends from the Doppler transducer 16 (not visible in FIG. 2) to a distal end carrying connectors 28, which comprise standard Doppler monitor connectors suitable to be received by a Doppler monitor.

Referring to FIG. 3, an exploded perspective view of the sensor of FIG. 2, the relative positions of sensor overmold 26, Doppler sensor 16 and adhesive conductive gel 18 are observed. In the illustrated embodiment, adhesive conductive gel 18 is a circular disk so as to be substantially co-extensive with the patient-side face portion of sensor overmold. In alternative embodiments, the adhesive conductive gel can be of different shape, or may be applied to the patient-side face portion separately prior to use.

Transducer 16 may comprise a piezoelectric ultrasonic transducer such as a model UZ250 sensor by Noliac (Noliac A/S of Denmark). Adhesive conductive gel 18 is suitably an adhesive conductive gel for attachment of the sensor to the patient, while enabling conduction of the Doppler signals from and to the transducer 16, and can comprise AG501 dermal fastener gel by AmGel Technologies of Fallbrook, Calif., or other adhesive compatible with Doppler signal transmission. Overmold 26 is suitably a EPDM (ethylene-propylene-diene monomer) polymer with a low durometer (e.g., 30 to 50). Exemplary materials for the overmold are provided under trade names Dutral brand Ethylene Propylene Copolymer by Polimeri Europa, Nordel brand by The Dow Chemical Company, and Vistalon brand by Exxon Mobil Corporation, for example. Connectors 28 are suitably standard AV connectors appropriate to be received by the Doppler monitor 22.

The overmold 26 may be provided in various shapes and configurations so as to fit or conform to the shape of the patient at the particular attachment location. The preferred and illustrated embodiment employs a semispherical shaped overmold 26. Other shapes, such as half egg-shaped may be employed. Also, the patient-side face is flat in the illustrated embodiment, but may also be contoured to conform more readily to the shape of the patient's body at a desired application position. All components that touch the patient are biocompatible.

Suitable dimensions of the sensor in particular embodiments are, 1 inch diameter circular disc or puck configuration, or, in the configuration shown in FIGS. 2 and 3, a 1 inch by inch semicircular dome having a dome with a inch height and having a 1 inch diameter at the base of the dome, the bottom face of the dome being a flat surface. The dome in FIGS. 2 and 3 is suitably radially symmetrical.

Procedures in which the device is useful include:

1) Arterial Sheath removal post Cardiac PTCA/Stent intervention while performing Manual Pressure or with the use of the Femstop Device.

2) Peripheral PTA/Stent intervention

3) Invasive lines (A-lines) including the use of IABP, Impella, LVAD, or CPS

4) Peripheral Surgery/procedures: Endarterectomy, Aorto/femoral bypass, Fem/Fem bypass, Fem/pop bypass.

In use, the device can be provided in a sterile package, which the medical personnel will open, to remove the device. Adhesive conductive gel 18 will have a removable protective covering thereover, such as a wax liner or the like, for protection prior to use. The covering is removed, and the exposed face of the gel is positioned on the patient at an appropriate location to enable sensing of blood flow. Connectors 28 are attached to a Doppler monitor 22, for continuous monitoring during the medical procedure. Once monitoring is no longer desired, the sensor portion is removed from the patient. The device may be provided in a single use pre-sterilized configuration, intended to be disposed of after use, or in a reusable configuration with adhesive replacement components provided for each use.

The illustrated use in FIG. 1 is on the patient's foot and dorsalis pedis artery, but other locations are also suitably monitored with the system and method, such as the leg and posterior tibial artery, or the arm and radial artery.

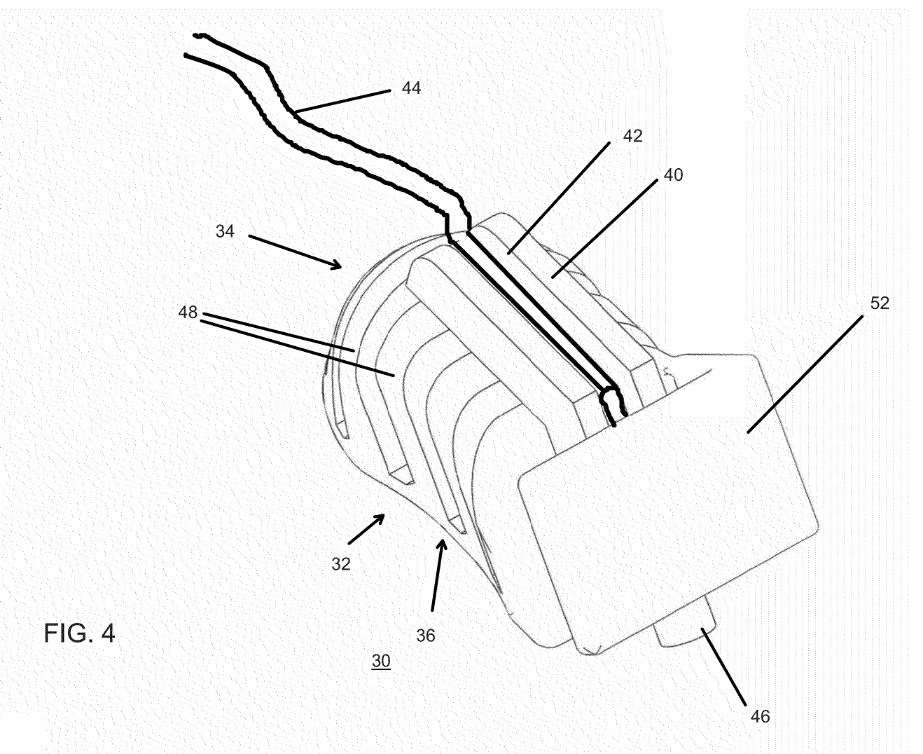

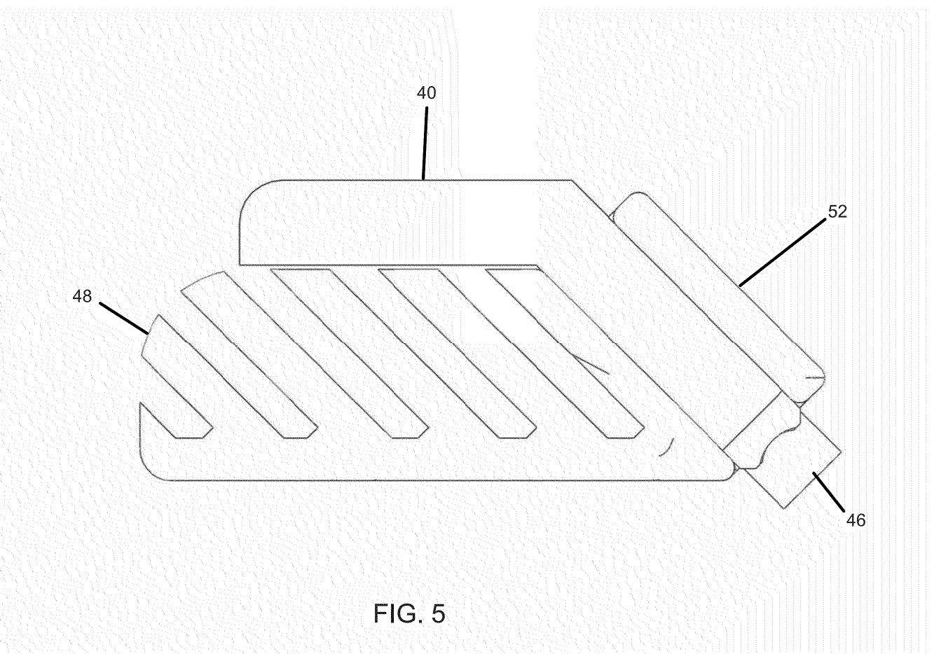

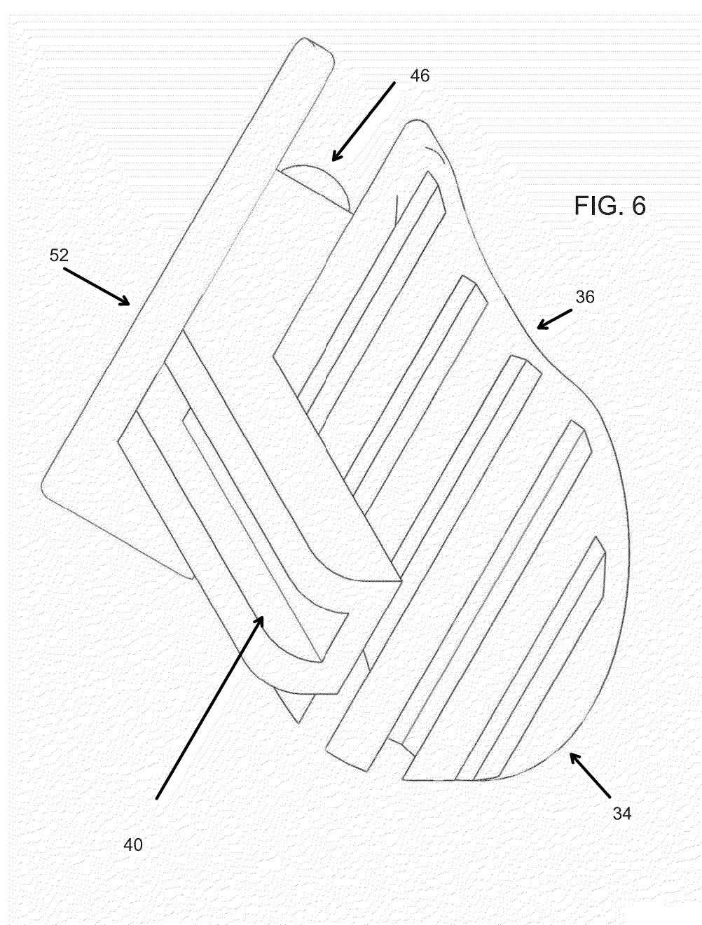

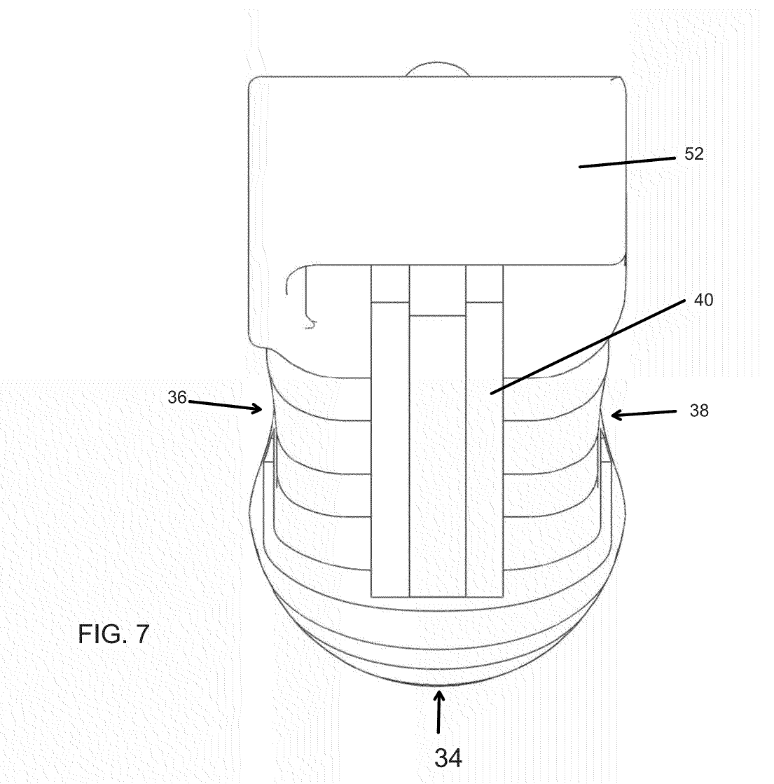

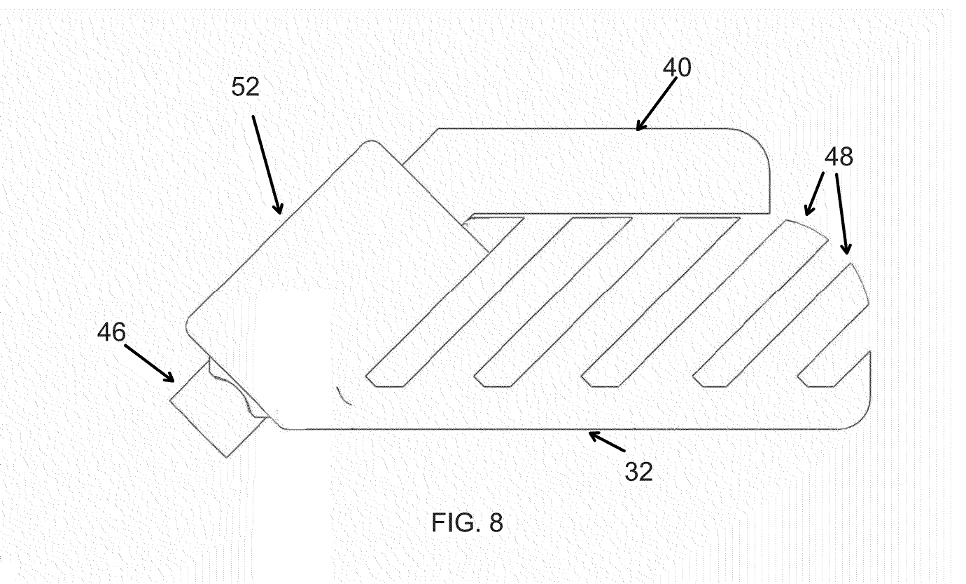

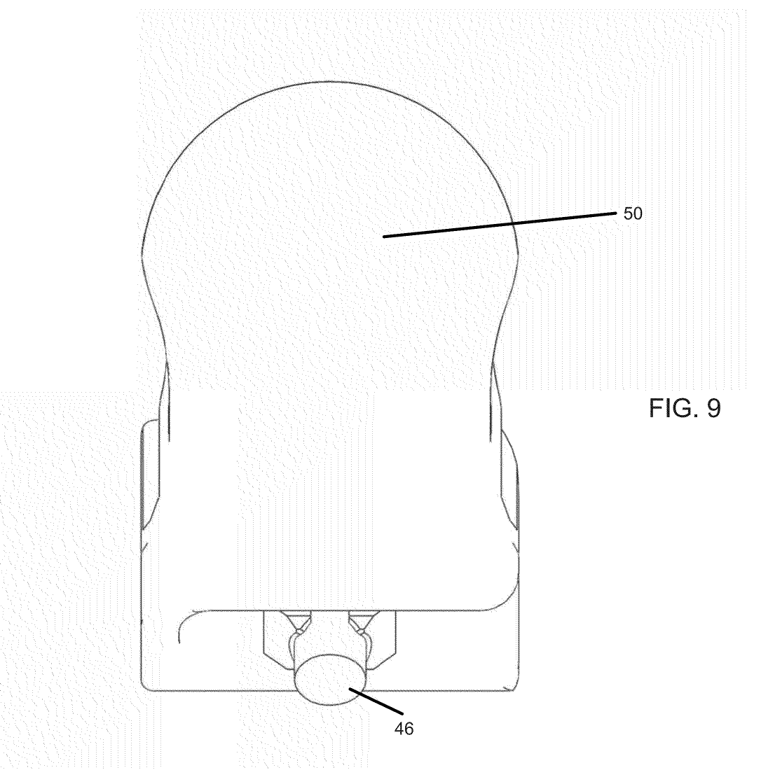

An embodiment of a holder for the Doppler probe is shown in FIGS. 4-9, which are front angled, side, rear top, top, left side and bottom views of an improved holder for a Doppler transducer. The holder 30 comprises a body 32 having a curved rear portion 34 and slightly concave left and right side portions 36, 38 for ease of gripping with a user's fingers. A top lead guide portion 40 extends from a front face of the holder approximately 75% of the length of the body, angling downwardly at the front face of the body. The guide portion includes a central channel 42 for receiving the sensor wiring 44 therein. A Doppler sensor 46 is mounted at an angle near the front face of the body, oriented downwardly to enable positioning on the patient's body. With reference to FIG. 9, the bottom face 50 of the holder is suitably smooth. Plural rib portions 48 may be formed as part of the body, whether for manufacturing reasons, such as in the case of manufacturing by injection molding, or for aesthetic reasons.

The front face 52 of the holder is substantially flat in the illustrated embodiment, and can provide a contact point for one or more of the user's fingers to assist in gripping the device, in conjunction with the concave side portions 36, 38.

FIG. 6 is a rear top view of the holder with the wire portion of the Doppler sensor removed, for illustration of the lead guide portion 40.

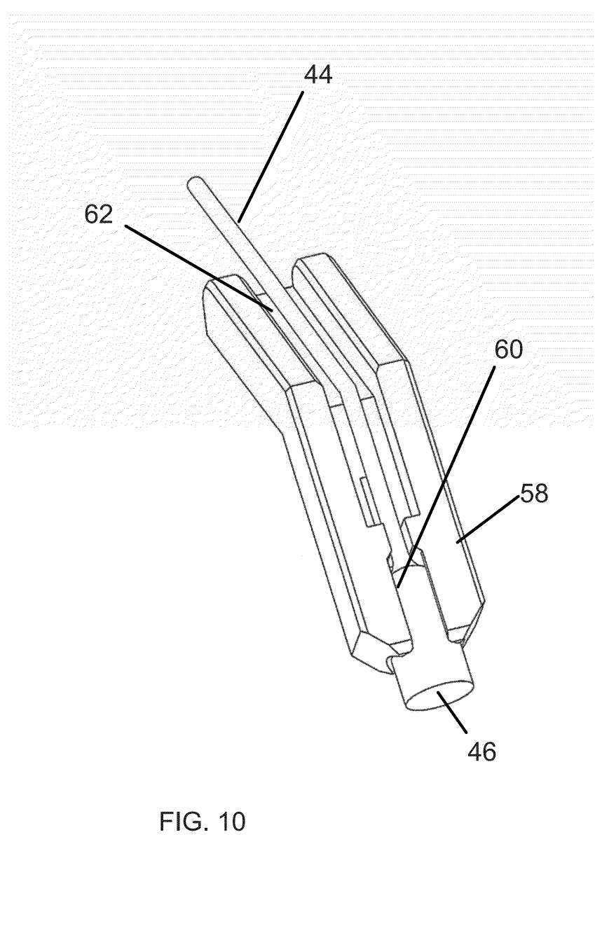

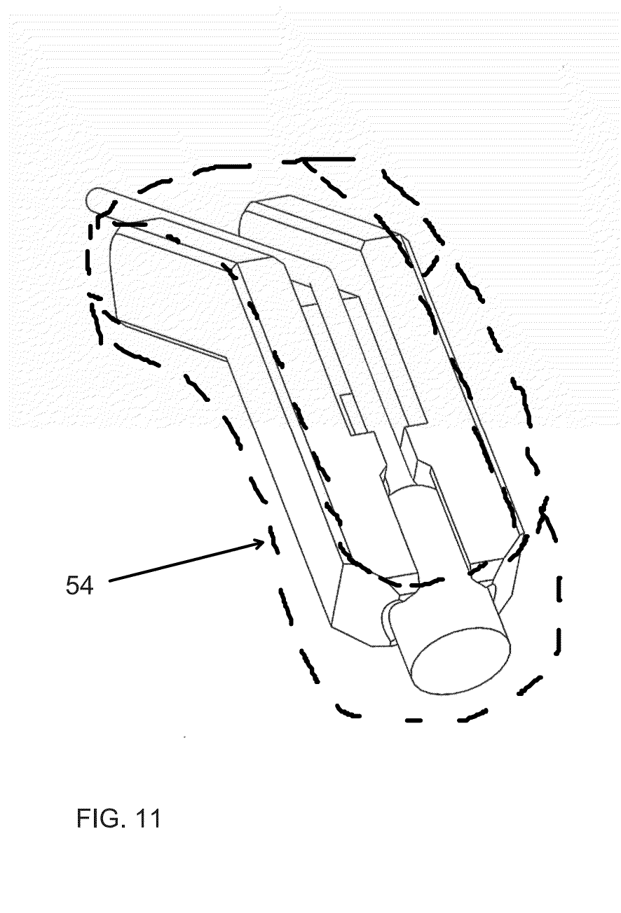

FIGS. 10 and 11 are two views of the sensor portion of the device, which is suitably removable by snapping into the holder, enabling replacement of the sensor/holder configuration. In a particular embodiment, the holder is a disposable item, with the sensor inserted into a sterile new holder for use, and removal of the sensor after use for disposal of the holder and recycling of the sensor after sterilization. The sensor portion may comprise a body 58 configured to fit within the holder body in a releasable manner, with the Doppler sensor 46 held in position therein by sensor engaging clips 60. A wire guide channel 62 carries the sensor wiring 44. In another manner of use, a condom type cover 54 may be place over the sensor before installation in the holder, for providing a sterile interaction with the patient. The holder may suitably be made of a moldable material such as plastic, manufactured at low cost for disposable use.

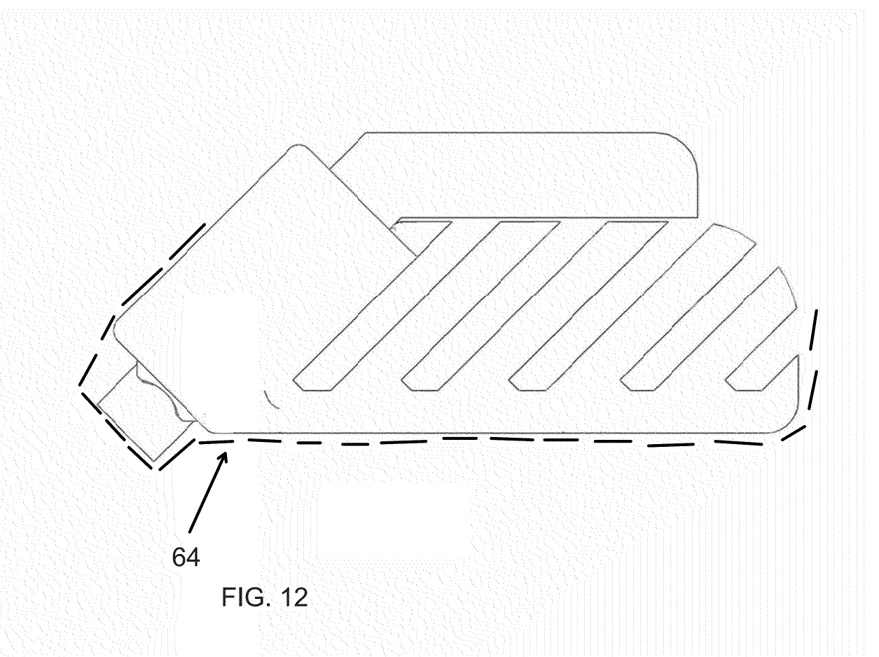

As an alternative to the condom cover 54, a barrier member 64 may be provided (FIG. 12) to prevent direct contact of the holder and Doppler sensor with the patient's body. The cover may be disposed of after each use, replaced with a new cover, which can be sterile, for each use of the device. In this configuration, both the sensor and the holder can be used on a more permanent basis, with only the cover being replaced after each use. The sensor and holder may be replaced at different schedules, as wear and procedures dictate.

Accordingly, a device comprising a small, compact, transducer assembly which is easily attached to the body is provided, to enable continuous blood flow monitoring via Doppler monitor at distal body parts over extended periods of time. Continuous Doppler monitoring enables medical personnel to ensure that blood flow is occurring at the distal body parts to verify that excessive pressure or no newly formed blood clots are occurring within the artery during a procedure. This is all accomplished in accordance with the system and method without requiring the MD/RN/tech to intermittently apply a Doppler device and constantly have to relocate the pulse.

While a preferred embodiment of the present invention has been shown and described, it will be apparent to those skilled in the art that many changes and modifications may be made without departing from the invention in its broader aspects. The appended claims are therefore intended to cover all such changes and modifications as fall within the true spirit and scope of the invention.

What is claimed is:

1. A sensor module for non-invasive continuous Doppler monitoring for arterial blood flow to a distal body part of a patient, comprising:
a holder body having,
at least one concave side portion adapted for conforming to a user's hand, and
a sensor receiving portion for removable placement of a sensor therein for use with a Doppler transducer.

2. The sensor module according to claim 1, further comprising a substantially flat front portion adapted for placement against a user's finger.

3. The sensor module according to claim 1, further comprising a barrier shield for placement over at least a portion of the holder body for providing a sterile barrier between the holder body and a patient's body.

4. The sensor module according to claim 1, further comprising a second concave side portion on a side of the holder body opposite said at least one concave side portion.

5. The sensor module according to claim 1, further comprising the sensor adapted for removably placing in the holder body for holding thereof by the holder body, and a sanitary cover member for providing a sterile barrier between the sensor and a patient's body.

6. The sensor module according to claim 1, wherein a patient-side face of said holder body is substantially flat.

7. The sensor module according to claim 1, wherein a patient-side face of said holder body is contoured to conform to a shape of a patient's body.

8. The sensor module according to claim 1, wherein the holder body is formed with plural rib portions extending across portions of the holder body.

9. A method for non-invasive continuous Doppler monitoring for arterial blood flow to a distal body part of a patient, comprising:
providing a holder body having,
at least one concave side portion adapted for conforming to a user's hand, and
a sensor receiving portion for removable placement of a sensor therein for use with a Doppler transducer.

10. The method according to claim 9, further comprising providing said holder body with a substantially flat front portion adapted for placement against a user's finger.

11. The method according to claim 9, further comprising providing a barrier shield for placement over at least a portion of the holder body for providing a sterile barrier between the holder body and a patient's body.

12. The method according to claim 9, further comprising said holder body having a second concave side portion on a side of the holder body opposite said at least one concave side portion.

13. The method according to claim 9, further comprising providing the sensor adapted for removably placing in the holder body for holding thereof by the holder body, and providing a sanitary cover member over the sensor for providing a sterile barrier between the sensor and a patient's body.

14. The method according to claim 9, wherein a patient-side face of said holder body is provided in a substantially flat configuration.

15. The method according to claim 9, wherein a patient-side face of said holder body is provided in a contoured form to conform to a shape of a patient's body

16. The method according to claim 9, wherein the holder body is formed with plural rib portions extending across portions of the holder body.
